SEQUENCE LISTING (1 sequence)

SEQ ID NO: 1 - DNA, 39 bp, synthetic construct
  misc_feature 1: note = amidated
  modified_base 1: mod_base = OTHER; note = 2'-Fluoro-cytidine
  modified_base 2: mod_base = gm
  modified_base 3..4: mod_base = OTHER; note = 2'-Fluoro-cytidine
  modified_base 6: mod_base = OTHER; note = 2'-Fluoro-cytidine
  modified_base 7..8: mod_base = gm
  modified_base 9: mod_base = OTHER; note = 2'-Fluoro-uridine
  modified_base 10: mod_base = OTHER; note = 2'-Fluoro-cytidine
  modified_base 11: mod_base = OTHER; note = 2'-Fluoro-uridine
  modified_base 12: mod_base = OTHER; note = 2'-Fluoro-cytidine
  modified_base 13: mod_base = OTHER; note = 2'-O-methyl-adenosine
  modified_base 14..15: mod_base = gm
  modified_base 16: mod_base = OTHER; note = 2'-Fluoro-cytidine
  modified_base 18: mod_base = OTHER; note = 2'-Fluoro-cytidine
  modified_base 19: mod_base = OTHER; note = 2'-Fluoro-uridine
  modified_base 20: mod_base = gm
  modified_base 21: mod_base = OTHER; note = 2'-O-methyl-adenosine
  modified_base 22: mod_base = gm
  modified_base 23: mod_base = OTHER; note = 2'-Fluoro-uridine
  modified_base 24: mod_base = OTHER; note = 2'-Fluoro-cytidine
  modified_base 25: mod_base = OTHER; note = 2'-Fluoro-uridine
  modified_base 26: mod_base = gm
  modified_base 27: mod_base = OTHER; note = 2'-O-methyl-adenosine
  modified_base 28: mod_base = gm
  modified_base 29..31: mod_base = OTHER; note = 2'-Fluoro-uridine
  modified_base 33..34: mod_base = OTHER; note = 2'-Fluoro-cytidine
  modified_base 35: mod_base = OTHER; note = 2'-Fluoro-uridine
  modified_base 36: mod_base = gm
  modified_base 37: mod_base = OTHER; note = 2'-Fluoro-cytidine
  modified_base 38: mod_base = gm
  misc_feature 38^39: note = 3' to 3' inverted phosphodiester linkage
  misc_feature 1..38: note = RNA fragment
  misc_feature 39: note = DNA fragment
  source 1..39: mol_type = other DNA
cgccgcggtctcaggcgctgagtctgagtttacctgcgt

(19) United States
(12) Patent Application Publication
(10) Pub. No.: US 2026/0185106 A1
(43) Pub. Date: Jul. 2, 2026

(54) PHARMACEUTICAL COMPOSITIONS FOR DELIVERY TO THE EYE

(71) Applicant: Astellas US LLC, Northbrook, IL (US)

(72) Inventors: Xiao-Ping DAI, Northbrook, IL (US); Eric N. GERSHENOW, Northbrook, IL (US); Keith WESTBY, Northbrook, IL (US); Glenn SBLENDORIO, Northbrook, IL (US); Wuming YAN, Northbrook, IL (US)

(21) Appl. No.: 19/413,886

(22) Filed: Dec. 9, 2025

Related U.S. Application Data
(63) Continuation of application No. 19/126,129.
(60) Provisional application No. 63/435,168, filed on Dec. 23, 2022.

(51) Int. Cl. C12N 15/115; A61K 9/19; A61K 47/60

(52) U.S. Cl. CPC C12N 15/115; A61K 9/19; A61K 47/60; C12N 2310/351

(57) ABSTRACT

Aptamers are single-stranded RNA or DNA molecules that bind to target molecules by forming complex three-dimensional structures. Their target molecules vary widely—including proteins, peptides, carbohydrates, lipids, small compounds, and metal ions—and because of their strong binding affinity and high specificity, aptamers have been put to practical use in fields such as therapeutics, diagnostics, and separation technologies. As pharmaceuticals, the world's first aptamer drug, Macugen®, was approved in the United States in 2004 as a treatment for neovascular age-related macular degeneration, and several other aptamers are now in clinical development. Research is also progressing on their use as tools in drug delivery systems (DDS), and the importance of aptamers in the pharmaceutical field is expected to continue growing. In this article, we provide an overview of aptamer acquisition methods and optimization strategies for therapeutic use, and introduce the latest developments in aptamer-based drugs currently in clinical trials.

### CROSS-REFERENCE TO RELATED APPLICATIONS

This application is a continuation of U.S. application Ser. No. 19/126,129, filed Apr. 30, 2025, which is a U.S. National Stage Application filed under 35 U.S.C. § 371 of International Application No. PCT/US2023/085312, filed Dec. 21, 2023, which claims the benefit of U.S. Provisional Patent Application No. 63/435,168, filed on Dec. 23, 2022, the disclosure of which are hereby incorporated by reference in their entirety.

### REFERENCE TO AN ELECTRONIC SEQUENCE LISTING

The contents of the electronic sequence listing (ASTL_005_02US_SeqList_ST26.xml; Size: 17,714 bytes; and Date of Creation: Dec. 9, 2025) are herein incorporated by reference in their entirety.

### FIELD OF THE DISCLOSURE

The presently disclosed subject matter relates to pharmaceutical compositions of avacincaptad pegol that are of sufficient purity to be suitable for administration to human patients in treating various ocular disorders and diseases.

### BACKGROUND OF THE DISCLOSURE

Disease and injury to tissues of the posterior segment of the eye, including the retina and choroid, is involved in many of the most common blinding diseases in the industrialized world. Age-related macular degeneration (AMD) alone impacts more than 10 million Americans. Severe vision loss from AMD and other diseases affecting the posterior segment, including diabetic retinopathy, glaucoma, and retinitis pigmentosa accounts for most cases of irreversible blindness worldwide. AMD is classified into one of two general subgroups; the non-neovascular (“dry”) form of the disease (“dry AMD”) and the neovascular form of the disease (“wet AMD”). Dry AMD is more prevalent, accounting for approximately 90% of all AMD cases. Dry AMD is characterized by the presence of drusen (yellow crystalline deposits that develop within the macula) located under the retinal pigment epithelial cells (RPE). When the condition is severe, dry AMD results in marked thinning and/or atrophy of the macula, resulting from the loss of the RPE and associated capillaries (choriocapillaris). This form of late-stage dry AMD is associated with thinning and loss of function of the neural retinal located above the affected RPE. This collective phenotype in late-stage dry AMD is termed geographic atrophy (“GA”). The progressive degeneration of light-sensitive photoreceptor cells in GA leads to severe visual loss in affected eyes. Currently, the treatment of posterior segment disease is to a significant extent limited by the difficulty in delivering effective doses of drugs to target tissues in the posterior eye.

Accordingly, despite significant efforts directed to treatment of GA, AMD or other ophthalmological disorders, there remains a scarcity of pharmaceutical compositions suitable for administration to human patients to treat ocular diseases requiring delivery of therapeutic agents to directly to the eye.

### SUMMARY

The presently disclosed subject matter provides highly purified drug substance and drug product of avacincaptad pegol (ACP). In one embodiment, the presently disclosed subject matter provides a composition comprising a non-pegylated aptamer intermediate having the sequence 5 NH₂-fCmGfCfCGfCmGmGfUfCfUfCmAmGmGfCGfCfUmGmAmGfUfCfUmGmAmGfUfUfUA fCfCfUmGf CmG-3T-3′ (SEQ ID NO: 1), wherein the composition has a purity profile as follows:
(a) more than 85% of the aptamer in the composition is full length aptamer;
(b) less than 3% total of fluoro degradants and n−1 deletion products; and
(c) 1% or less of deprotection failure products.
   In some embodiments the composition additionally has:
(d) less than 1.8% G cleavage product;
(e) less than 0.1% A cleavage product; and
(f) less than 1.1% total of n−4, n−3 and n−2 deletion products.
   In some embodiments, the presently disclosed subject matter provides a PEGylated aptamer comprising the ultrapure compositions in the embodiments above, having the structure:

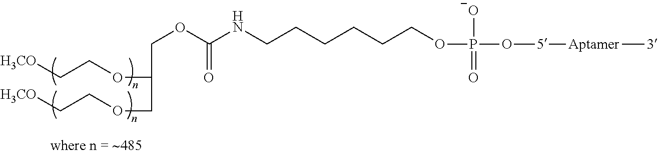

or a salt thereof.

In another embodiment, the presently disclosed subject matter provides an ultrapure drug substance comprising avacincaptad pegol wherein the drug substance has a purity profile as follows:
(a) more than 92% of the aptamer in the drug substance is full length aptamer;
(b) less than 1.5% of the drug substance is relative retention time (RRT) 1 (≥0.93-<full length product (FLP)); and
(c) less than 5% of the drug substance is RRT2 (>FLP-≤1.2).

In another embodiment, the presently disclosed subject matter provides a drug substance wherein the potency of the drug substance, as measured by ELISA, is greater than 95%.

In another embodiment, the presently disclosed subject matter provides a pharmaceutical composition comprising the ultrapure drug substance described herein, and one or more pharmaceutically acceptable excipients. In some embodiments, the pharmaceutical composition is formulated at a concentration of 20 mg/mL (oligonucleotide mass) in phosphate-buffered saline at pH 6.8-7.8 as a sterile aqueous solution. In some embodiments, the pharmaceutical composition has an osmolality between 350-500 mOsM/kg.

In another embodiment, the presently disclosed subject matter provides methods for treating an ophthalmological disease, disorder, and/or condition comprising intravitreally administering the ultrapure Avacincaptad pegol pharmaceutical composition described herein at a dose of between 3-5 mg/eye to a subject in need thereof. In a preferred embodiment, the method of the presently disclosed subject matter provides that the dose administered is about 2 mg/eye.

### BRIEF DESCRIPTION OF THE DRAWINGS

FIG. 1 depicts pre- and post-FLP impurity fraction collections from a purification process for PEGylated ACP.

### DETAILED DESCRIPTION OF THE DISCLOSURE

Provided herein is a highly purified drug substance and drug product comprising avacincaptad pegol (ACP). These compositions are produced by an improved manufacturing process. The highly purified compositions are advantageous compared to compositions produced by a prior art process, at least in part because the highly purified compositions contain a lower proportion of ACP impurities or variants that do not contribute to efficacy but are available to induce an immune response.

As used herein, including the appended claims, the singular forms of words such as “a,” “an,” and “the,” include their corresponding plural references unless the context clearly dictates otherwise. All references cited herein are incorporated by reference to the same extent as if each individual publication, patent application, or patent, was specifically and individually indicated to be incorporated by reference.

### Definitions

Throughout this application, the term “about” is used to indicate that a value includes the inherent variation of error for the device or the method being employed to determine the value, or the variation that exists among the samples being measured. Unless otherwise stated or otherwise evident from the context, the term “about” means within 10% above or below the reported numerical value (except where such number would exceed 100% of a possible value or go below 0%). When used in conjunction with a range or series of values, the term “about” applies to the endpoints of the range or each of the values enumerated in the series, unless otherwise indicated. As used in this application, the terms “about” and “approximately” are used as equivalents.

The term “ocular” as used in the presently disclosed subject matter refers to the eye in general, or any part or portion of the eye (as an “ocular implant” according to the presently disclosed subject matter can in principle be administered to any part or portion of the eye) or any disease of the eye (as in one aspect the presently disclosed subject matter generally refers to treating any diseases of the eye (“ocular diseases”), of various origin and nature. The presently disclosed subject matter in certain embodiments is directed to intravitreal injection of an ocular implant (in this case the “ocular implant” is thus an “intravitreal implant”), and to the treatment of ocular diseases affecting the posterior segment of the eye, as further disclosed below.

The term “patient” herein includes both human and animal patients. A “patient” is a subject in need of treatment due to a particular physiological or pathological condition.

The term “polymer network” describes a structure formed of polymer chains (of the same or different molecular structure and of the same or different molecular weight) that are crosslinked with each other. The types of polymers suitable for the purposes of the presently disclosed subject matter are disclosed herein. The polymer network may also be formed with the aid of a crosslinking agent as also disclosed herein,

Open terms such as “include,” “including,” “contain,” “containing” and the like as used herein mean “comprising” and are intended to refer to open-ended lists or enumerations of elements, method steps, or the like and are thus not intended to be limited to the recited elements, method steps or the like but are intended to also include additional, unrecited elements, method steps or the like.

The term “up to” when used herein together with a certain value or number is meant to include the respective value or number. When ranges of values or numbers are used herein, the endpoints of the range are included in the range.

The terms “API”, “active (pharmaceutical) ingredient”, “active (pharmaceutical) agent”, “active (pharmaceutical) principle”, “(active) therapeutic agent”, “active”, “drug”, and “drug substance” are used interchangeably herein and refer to the substance used in a finished pharmaceutical product (FPP) or “drug product”, as well as the substance used in the preparation of such a finished pharmaceutical product intended to furnish pharmacological activity or to otherwise have direct effect in the diagnosis, cure, mitigation, treatment or prevention of a disease, or to have direct effect in restoring, correcting or modifying physiological functions in a patient.

The term “aptamer” as used herein refers to an oligonucleotide and/or nucleic acid analogues that can bind to a specific target molecule. Aptamers can include RNA, DNA, RNA/DNA, any nucleic acid analogue, and/or combinations thereof. Aptamers can be single-stranded oligonucleotides. Without wishing to be bound by theory, aptamers are thought to bind to a three-dimensional structure of a target molecule. Aptamers may be monomeric (composed of a single unit) or multimeric (composed of multiple units). Multimeric aptamers can be homomeric (composed of multiple identical units) or heteromeric (composed of multiple non-identical units).

### Drug Substance

The drug substance is a pegylated anti-C5 agent which is an Aptamer=5′ NH₂-fCmGfCfCGfCmGmGfUfCfUfCmAmGmGfCGfCfUmGmAmGfUfCfUmGmAmGfUfUfUA fCfCfUmGf CmG-3T-3′, (SEQ ID NO: 1), wherein fC and fU=2′ fluoro nucleotides, mG and mA=2′-OMe nucleotides, G and A=ribonucleotides, and 3T indicates an inverted deoxythymidine. The structure is shown below:

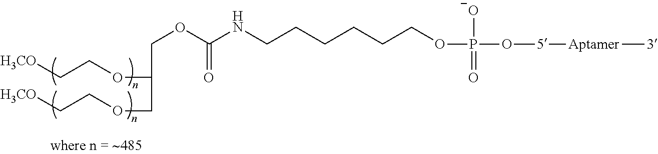

ARC1905 (avacincaptad pegol) is a PEGylated RNA aptamer which is a potent and specific inhibitor of complement activation that is being developed as a therapy for the treatment of age-related macular degeneration (AMD), geographic atrophy (GA) secondary to AMD, and Stargardt disease. The molecular formula of avacincaptad pegol (free acid form) is C₃₉₅H₄₉₂N₁₄₂O₂₆₂P₃₉F₂₁ ((CH₂)₂O)ₙ where n˜970 and the molecular weight is approximately 56 kDa. ARC1905 consists of a 12,882 Dalton modified RNA aptamer that is conjugated at the 5′ terminus to a polyethylene glycol (PEG) moiety. The aptamer portion of ARC1905 (ARC672) is 39 nucleotides in length and modified with a primary amine at the 5′ terminus to provide a reactive site for site specific conjugation (“PEGylation”). Specifically, ARC1905 is a PEGylated aptamer containing 39 monomeric units (39-mer) with a hairpin structure. Certain activated PEG moieties for conjugation to the above-referenced RNA aptamer are commercially available. Other activated PEG moieties can be prepared using known methods. It is understood that the PEG moieties used in the presently disclosed subject matter are a collection of individual PEG molecules of varying molecular weight. Further, PEG moieties are generally characterized by a numeric description that indicates the average molecular weight of the PEG polymers contained therein. For example, a 40 kDa PEG moiety generally refers to a PEG moiety having an average molecular weight of about 40 kDa. In some embodiments, the PEG portion of ARC1905 is a 2-arm branched PEG. In some embodiments, the PEG portion of ARC1905 is a 2-arm branched PEG having an average molecular weight ranging from approximately 39 kDa to approximately 47 kDa (including endpoints of the range). In some embodiments, the PEG portion of ARC1905 is a 2-arm branched PEG having an average molecular weight of approximately 40 kDa. In other embodiments, the PEG portion of ARC1905 is a 2-arm branched PEG having an average molecular weight of approximately 43 kDa. In some embodiments, the PEG portion of ARC1905 is a 2-arm branched NHS carbonate PEG. In some embodiments, the PEG reagent for ARC1905 is SUNBRIGHT® GL2-400TS (2-arm branched NHS carbonate PEG) (NOF America Corporation). According to the manufacturer's specifications, SUNBRIGHT® GL2-400TS has an average molecular weight (Mp) range of 39-47 kDa. In some embodiments, the average molecular weight (Mp) of mPEG2-NHS ester, as determined by gel permeation chromatography (GPC), is 39-47 kDa. Other suitable PEG reagents include, without limitation, SUNBRIGHT® GL2-400NP, ME-200TS, ME-300TS, ME-400TS, ME-400HS, ME-400GS, ME-400CS, GL2-200TS, GL2-600TS, or LY-400NS (NOF America Corporation).

The nucleotide composition consists of ribo purines and modified 2′-fluoro pyrimidines and 2′-methoxy purines. The modified nucleotides minimize susceptibility to endonuclease digestion. The 3′ terminus is capped with an “inverted” 3′-3′ phosphodiester linkage to a deoxythymidine nucleotide (idT) to maximize resistance to 3′-exonuclease degradation. PEGylation is employed to improve lifetime in vivo without diminishing affinity or activity. ARC1905 aptamer forms a hairpin structure with a functionally important internal asymmetric bulge, an internal loop, and terminal hairpin loop.

ARC1905 inhibits C5, a central component of the complement cascade, which plays multiple roles in innate immunity and inflammatory diseases. ARC1905 binds to human C5 with high specificity and affinity (K_{D}=0.69±0.148 nM at 37° C.) and is a potent inhibitor of C5 resulting from the activation of the classical and alternate complement (C′) pathways.

The drug substances, drug product, and compositions provided herein comprise avacincaptad pegol. In this specification, “avacincaptad pegol” or “ACP” refers either to the form of its free base or the form of its salt. In some embodiments, avacincaptad pegol may be present in the form of its salt. In some embodiments, a salt of avacincaptad pegol is a pharmaceutically acceptable salt of avacincaptad pegol. In a preferred embodiment, a salt of avacincaptad pegol is a sodium salt of avacincaptad pegol. In some embodiments, a salt of avacincaptad pegol is, for example, an alkali metal salt such as sodium salt, potassium salt, lithium salt and the like. In some embodiments, a salt of avacincaptad pegol is, for example, an alkaline earth metal salt such as calcium salt, magnesium salt and the like. In some embodiments, salts of avacincaptad pegol include, without limitation, salts with organic bases such as triethylamine, dicyclohexylamine, pyrrolidine, morpholine, pyridine and the like; ammonium salts and the like. In some embodiments, salts of avacincaptad pegol include, without limitation, inorganic acids such as hydrochloric acid, hydrobromic acid, sulfuric acid, nitric acid, phosphoric acid and the like; and salts with organic acids such as acetic acid, oxalic acid, citric acid, lactic acid, tartaric acid, p-tolu-enesulfonic acid and the like.

Examples of a pharmaceutically acceptable salt include, but are not limited to, sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, pamoate, phenylacetate, trifluoroacetate, acrylate, chlorobenzoate, dinitrobenzoate, hydroxybenzoate, methoxybenzoate, methylbenzoate, o-acetoxybenzoate, naphthalene-2-benzoate, isobutyrate, phenylbutyrate, a-hydroxybutyrate, butyne-1,4-dicarboxylate, hexyne-1,4-dicarboxylate, caprate, caprylate, cinnamate, glycollate, heptanoate, hippurate, malate, hydroxymaleate, malonate, mandelate, mesylate, nicotinate, phthalate, teraphthalate, propiolate, propionate, phenylpropionate, sebacate, suberate, p-bromobenzenesulfonate, chlorobenzenesulfonate, ethylsulfonate, 2-hydroxyethylsulfonate, methylsulfonate, naphthalene-1-sulfonate, naphthalene-2-sulfonate, naphthalene-1,5-sulfonate, xylenesulfonate, and tartarate salts. The term “pharmaceutically acceptable salt” includes, but is not limited to, a hydrate of avacincaptad pegol and also may refer to a salt of avacincaptad pegol having an acidic functional group, such as, but not limited to, a carboxylic acid functional group or a hydrogen phosphate functional group, and a base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or tri-alkylamines, dicyclohexylamine; tributyl amine; pyridine; N-methyl, N-ethylamine; diethylamine; triethylamine; mono-, bis-, or tris-(2-OH-lower alkylamines), such as mono-, bis-, or tris-(2-hydroxyethyl)amine, 2-hydroxy-tert-butylamine, or tris-(hydroxymethyl)methylamine; N,N-di-lower alkyl-N-(hydroxyl-lower alkyl)-amines, such as N,N-dimethyl-N-(2-hydroxyethyl)amine or tri-(2-hydroxyethyl)amine; N-methyl-D-glucamine; and amino acids such as arginine, lysine, and the like.

In some embodiments, the presently disclosed subject matter provides a composition comprising a non-pegylated aptamer intermediate having the sequence 5′ NH₂-fCmGfCfCGfCmGmGfUfCfUfCmAmGmGfCGfCfUmGmAmGfUfCfUmGmAmGfUfUfUA fCfCfUmGf CmG-3T-3′ (SEQ ID NO: 1), wherein the composition has a purity profile as follows:
(a) more than 85% of the aptamer in the composition is full length aptamer;
(b) less than 3% total of fluoro degradants and n−1 deletion products; and
(c) 1% or less of deprotection failure products.
   In some embodiments the composition additionally has:
(d) less than 1.8% G cleavage product;
(e) less than 0.1% A cleavage product; and
(f) less than 1.1% total of n−4, n−3 and n−2 deletion products.
   In some embodiments, the presently disclosed subject matter provides a PEGylated aptamer comprising the ultrapure compositions in the embodiments above, having the structure:

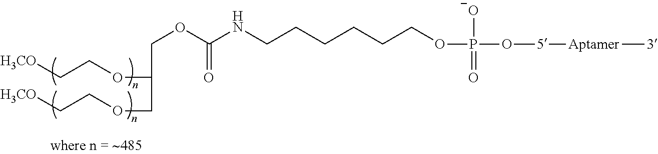

or a salt thereof.

In some embodiments, an ultrapure drug substance comprising avacincaptad pegol is generated, wherein the drug substance has the following characteristics:
(a) more than 92% of the aptamer in the drug substance is full length aptamer;
(b) less than 1.5% of the drug substance is RRT1 (>0.93-<FLP); and
(c) less than 5% of the drug substance is RRT2 (>FLP-≤1.2).

Potency of the ultrapure intermediate and drug substance can be measured by ELISA. The ELISA assay is based on induction of complement cascade by Lipopolysaccharide (LPS) and quantification of C5b9 formation. The higher the potency that ARC1905 has, the lower the amount of C5b9 that is detected. The result is expressed as relative potency (%) compared to standard ARC1905 reference material.

Based on the ELISA results, the ultrapure drug substance was at least 5% more potent compared to product made according to the prior art synthesis. In some embodiments, the ultrapure drug substance has a potency of the drug substance, as measured by ELISA, is greater than 95%.

In some embodiments, the content of endotoxin in the ultrapure drug substance is less than 0.2 EU/dose. In one embodiment, the content of endotoxin in the drug product is about 0.14 EU/dose, preferably about 0.05 EU/dose.

Exemplary methods for producing the ultrapure drug substance are provided in Example 2.

### Drug Product

ARC1905 drug product is a preservative-free, sterile, aqueous solution for intravitreal injection. It is formulated at a concentration of 20 mg/mL (oligonucleotide mass) in phosphate-buffered saline at pH 6.8-7.8 as a sterile aqueous solution. The drug product is presented in a 2.0 mL clear, Type I glass vial, stoppered with a rubber stopper and sealed with an aluminum seal with flip-off cap. The drug product is stable at 2-8° C. for 43 months.

The osmolality (as measured by freezing point depression) of ARC1905 at a concentration of 20 mg/mL has a range of between 350-500 mOsM/kg, preferably between 400-450 mOsM/kg (including endpoints of the range).

### Administration and Dosage

ARC1905 is intended for dosing by intravitreal injection at between 1-5 mg/eye per administration. In some embodiments, dosing by intravitreal injection is preferably 2 mg/eye per administration. In some embodiments, dosing by intravitreal injection is preferably 4 mg/eye per administration (in one or more injections per eye on the same patient visit). Dosing can be biweekly, monthly, every other month, or quarterly. In some embodiments, the dosing can be once monthly. In some embodiment, the dosing can be once monthly for up to 12 months. In some embodiments, the dosing can be approximately every 28±7 days.

In some embodiments, a dosing regimen comprising a loading phase and maintenance phase may be administered.

In some embodiments, avacincaptad pegol or a salt thereof may be administered in a dosing regimen comprising a loading phase that comprises a dose of about 2 mg/eye administered once a month for a duration of up to one year, followed by a maintenance phase that comprises a dose of the avacincaptad pegol of about 0.3 mg/eye, or about 0.5 mg/eye, or about 0.75 mg/eye, or about 1 mg/eye, or about 1.25 mg/eye, or about 1.50 mg/eye, or about 1.75 mg/eye, or about 2 mg/eye, or about 2.25 mg/eye, or about 2.50 mg/eye, or about 2.75 mg/eye, or about 3 mg/eye, or about 3.25 mg/eye, or about 3.50 mg/eye, or about 3.75 mg/eye, or about 4 mg/eye, administered once every 8 weeks, once every 9 weeks, once every 10 weeks, once every 11 weeks, once every 12 weeks, once every 13 weeks, once every 14 weeks, once every 14 weeks, once every 15 weeks, once every 16 weeks, once every 17 weeks, once every 18 weeks, once every 19 weeks, once every 20 weeks, once every 21 weeks, once every 22 weeks, once every 23 weeks, once every 24 weeks, once every 25 weeks, or once every 26 weeks.

### Methods of Use

The drug product described herein is suitable for use in any of the methods of the presently disclosed subject matter described herein.

In some embodiments, the presently disclosed subject matter is a method of treating a disease or disorder of the eye in a subject in need thereof, comprising administering the drug product of the presently disclosed subject matter to an ocular area of the subject.

As used herein, the terms “treat”, “treatment” and “treating” refer to therapeutic treatments includes the reduction or amelioration of the progression, severity and/or duration of a disease, disorder or condition, or the amelioration of one or more symptoms (specifically, one or more discernible symptoms) of a disease, disorder or condition, resulting from the administration of the compositions or implant of the presently disclosed subject matter. In specific embodiments, the therapeutic treatment includes the amelioration of at least one measurable physical parameter of a disease, disorder or condition. In other embodiments the therapeutic treatment includes the inhibition of the progression of a condition, either physically by, e.g., stabilization of a discernible symptom, physiologically by, e.g., stabilization of a physical parameter, or both. In other embodiments the therapeutic treatment includes the reduction or stabilization of a disease, disorder or condition.

In some embodiments, the ocular disease refers to any disease that is impacting retina, retinal pigment epithelium (RPE) and choroid. In some embodiments, the ocular disease is selected from the group consisting of: geographic atrophy secondary to age-related macular degeneration (AMD), dry age-related macular degeneration (dry AMD), wet age-related macular degeneration (wet AMD), neovascular age-related macular degeneration (nAMD), retinal vein occlusion (RVO), diabetic macular edema (DME), diabetic retinopathy (DR), Usher syndrome type 1, Usher syndrome type 2, Usher syndrome type 3, Stargardt disease, uveitis, red-green color blindness, blue cone monochromacy, Leber congenital amaurosis (LCA), Leber hereditary optic neuropathy (LHON), neuromyelitis optica (NMO), choroideremia, X-linked retinoschisis (XLRS), Bardet-Biedl syndrome, cone dystrophy, optic atrophy, retinitis pigmentosa, age-related retinal ganglion cell (RGC) degeneration, Best disease, glaucoma, Graves' ophthalmopathy, multiple sclerosis (MS)-associated vision loss, myopia, X-linked recessive ocular albinism, oculocutaneous albinism type 1, optic neuritis, polypoidal choroidal vasculopathy, X-linked retinitis pigmentosa (XLRP), achromatopsia (ACHM), biallelic RPE65 mutation-associated retinal dystrophy, idiopathic polypoidal choroidal vasculopathy, high-risk drusen, a condition selected from the group consisting of risk factors for the progression to iRORA (incomplete RPE and outer retinal atrophy), iRORA, nascent geographic atrophy (nGA), and cRORA (complete RPE and outer retinal atrophy). In preferred embodiments, the ocular disease is geographic atrophy secondary to AMD, or autosomal recessive Stargardt Disease (STGD1). In some embodiments, the ocular disease is geographic atrophy secondary to AMD.

References to methods of treatment in this description are to be interpreted as references to compounds, pharmaceutical compositions and medicaments of the presently disclosed subject matter for use in those methods.

In some embodiments, provided herein is a drug substance or a pharmaceutical composition disclosed herein for use as a medicament. In some embodiments, provided herein is a drug substance or a pharmaceutical composition disclosed herein for in the treatment of an ophthalmological disease, disorder, and/or condition.

The drug product may be used as a monotherapy or in combination with a second suitable ocular therapy. In preferred embodiments, the second ocular therapy is a VEGF antagonist, such as aflibercept, ranibizumab, bevacizumab or faricimab.

### NUMBERED EMBODIMENTS

Notwithstanding the appended claims, the disclosure sets forth the following numbered embodiments:
Embodiment 1. A composition comprising the non-pegylated aptamer 5′ NH₂-fCmGfCfCGfCmGmGfUfCfUfCmAmGmGfCGfCfUmGmAmGfUfCfUmGmAmGfUfUfUA fCfCfUmGf CmG-3T-3′ (SEQ ID NO: 1), wherein the composition comprises:
(a) more than 85% of the aptamer in the composition is full length aptamer;
(b) less than 1.8% G cleavage product; and
(c) 1% or less of deprotection failure products.
Embodiment 2. The composition of embodiment 1, wherein the composition additionally has:
(d) less than 0.1% A cleavage product;
(e) less than 1.1% total of n−4, n−3 and n−2 deletion products; and
(f) less than 3% total of fluoro degradants and n−1 deletion products.
Embodiment 3. A PEGylated aptamer comprising the composition of embodiment 1 or 2 that has the structure:

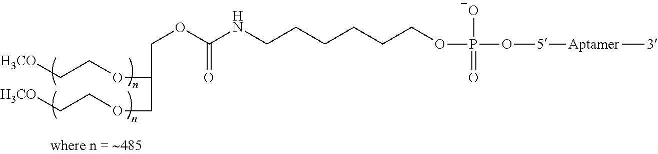

or a salt thereof.
Embodiment 4. The PEGylated aptamer of embodiment 3, wherein the PEGylated aptamer comprises a 2-arm branched ranging from approximately 39 kDa to approximately 47 kDa PEG.
Embodiment 5. The PEGylated aptamer of embodiment 3, wherein the PEGylated aptamer comprises a 2-arm branched approximately 40 kDa PEG.
Embodiment 6. The PEGylated aptamer of embodiment 3, wherein the PEGylated aptamer comprises a 2-arm branched approximately 43 kDa PEG.
Embodiment 7. The PEGylated aptamer of any one of embodiments 3-6, wherein the salt is a sodium salt.
Embodiment 8. A drug substance comprising avacincaptad pegol thereof wherein the drug substance comprises:
(a) more than 92% of the aptamer in the drug substance is full length aptamer;
(b) less than 1.5% of the drug substance is relative retention time (RRT) 1 (≥0.93-<full length product (FLP)); and
(c) less than 5% of the drug substance is RRT2 (>FLP-≤1.2).
Embodiment 9. The drug substance of embodiment 8, wherein the drug substance comprises a sodium salt of avacincaptad pegol.
Embodiment 10. The drug substance of embodiment 8 or 9, wherein the potency of the drug substance, as measured by ELISA, is greater than 95%.
Embodiment 11. A pharmaceutical composition comprising the drug substance of any one of embodiments 8-10 and one or more pharmaceutically acceptable excipients.
Embodiment 12. The pharmaceutical composition of embodiment 11, wherein the composition is formulated at a concentration of 20 mg/mL (oligonucleotide mass) in phosphate-buffered saline at pH 6.8-7.8 as a sterile aqueous solution.
Embodiment 13. The pharmaceutical composition of embodiment 12, wherein the composition has an osmolality between 350-500 mOsM/kg.
Embodiment 14. A method for treating an ophthalmological disease, disorder, and/or condition, the method comprising intravitreally administering the pharmaceutical composition of any one of embodiments 6-8 at a dose of between 3-5 mg/eye to a subject in need thereof.
Embodiment 15. The method of embodiment 14, wherein the dose administered is about 2 mg/eye.
Embodiment 16. The method of embodiment 15, wherein 100 μL is injected per eye.
Embodiment 17. The method of any one of embodiments 14-16, wherein the ophthalmological disease, disorder, and/or condition is selected from the group consisting of geographic atrophy secondary to age-related macular degeneration, dry age-related macular degeneration (dry AMD), wet age-related macular degeneration (wet AMD), neovascular age-related macular degeneration (nAMD), retinal vein occlusion (RVO), diabetic macular edema (DME), diabetic retinopathy (DR), Usher syndrome type 1, Usher syndrome type 2, Usher syndrome type 3, Stargardt disease, uveitis, red-green color blindness, blue cone monochromacy, Leber congenital amaurosis (LCA), Leber hereditary optic neuropathy (LHON), neuromyelitis optica (NMO), choroideremia, X-linked retinoschisis (XLRS), Bardet-Biedl syndrome, cone dystrophy, optic atrophy, retinitis pigmentosa, age-related retinal ganglion cell (RGC) degeneration, Best disease, glaucoma, Graves' ophthalmopathy, multiple sclerosis (MS)-associated vision loss, myopia, X-linked recessive ocular albinism, oculocutaneous albinism type 1, optic neuritis, polypoidal choroidal vasculopathy, X-linked retinitis pigmentosa (XLRP), achromatopsia (ACHM), biallelic RPE65 mutation-associated retinal dystrophy, idiopathic polypoidal choroidal vasculopathy, high-risk drusen, and a condition selected from the group consisting of risk factors for the progression to iRORA (incomplete RPE and outer retinal atrophy), iRORA, nascent geographic atrophy (nGA), and CRORA (complete RPE and outer retinal atrophy).
Embodiment 18. The method of any one of embodiments 14-17, wherein the dose is administered once monthly.
Embodiment 19. The method of any one of embodiments 14-18, wherein the dose is administered once monthly for up to 12 months.
Embodiment 20. The drug substance of any one of embodiments 8-10 or the pharmaceutical composition of any one of embodiments 11-13 for use as a medicament.
Embodiment 21. The drug substance of any one of embodiments 8-10 or the pharmaceutical composition of any one of embodiments 11-13 for use in the treatment of an ophthalmological disease, disorder, and/or condition.
Embodiment 22. The drug substance or the pharmaceutical composition for use of embodiment 21, wherein the ophthalmological disease, disorder, and/or condition is selected from the group consisting of geographic atrophy secondary to age-related macular degeneration, dry age-related macular degeneration (dry AMD), wet age-related macular degeneration (wet AMD), neovascular age-related macular degeneration (nAMD), retinal vein occlusion (RVO), diabetic macular edema (DME), diabetic retinopathy (DR), Usher syndrome type 1, Usher syndrome type 2, Usher syndrome type 3, Stargardt disease, uveitis, red-green color blindness, blue cone monochromacy, Leber congenital amaurosis (LCA), Leber hereditary optic neuropathy (LHON), neuromyelitis optica (NMO), choroideremia, X-linked retinoschisis (XLRS), Bardet-Biedl syndrome, cone dystrophy, optic atrophy, retinitis pigmentosa, age-related retinal ganglion cell (RGC) degeneration, Best disease, glaucoma, Graves' ophthalmopathy, multiple sclerosis (MS)-associated vision loss, myopia, X-linked recessive ocular albinism, oculocutaneous albinism type 1, optic neuritis, polypoidal choroidal vasculopathy, X-linked retinitis pigmentosa (XLRP), achromatopsia (ACHM), biallelic RPE65 mutation-associated retinal dystrophy, idiopathic polypoidal choroidal vasculopathy, high-risk drusen, and a condition selected from the group consisting of risk factors for the progression to iRORA, iRORA, nGA, and cRORA.

The following examples are provided only for illustrative purposes and are not intended to limit the invention in any way.

### EXAMPLES

### Example 1: Avacincaptad Pegol Synthesis According to Prior Art Method

The oligonucleotide 5′ NH₂-fCmGfCfCGfCmGmGfUfCfUfCmAmGmGfCGfCfUmGmAmGfUfCfUmGmAmGfUfUfUA fCfCfUmGf CmG-3T-3′ (SEQ ID NO: 1), was synthesized on an Expedite DNA synthesizer (ABI, Foster City, Calif.) according to the recommended manufacturer's procedures using Standard commercially available 2′-OMe RNA and 2′-F RNA and TBDMS-protected RNA phosphoramidites (Glen Research, Sterling, Va.) and an inverted deoxythymidine CPG Support. Terminal amine function was attached with a 5′-amino modifier C6-TFA (Glen Research, Sterling, VA). After deprotection, the oligonucleotides were purified by ion exchange chromatography on Super Q 5 PW (30) resin (ToSoh BioSciences) and ethanol precipitated.

The amine-modified aptamer was conjugated to different PEG moieties post-synthetically. The aptamer was dissolved in a water/DMSO (1:1) solution to a concentration between 1.5 and 3 mM. Sodium carbonate buffer, pH 8.5, was added to a final concentration of 100 mM, and the oligo was reacted overnight with a 1.7 molar excess of the desired PEG reagent (e.g., SUNBRIGHT® GL2-400NP, SUNBRIGHT® GL2-400TS (NOF Corp, Japan), or ARC187 40 kDa mPEG2-NHS ester (Nektar, Huntsville, Ala.)) dissolved in an equal volume of acetonitrile. The resulting products were purified by ion exchange chromatography on Super Q 5 PW (30) resin (Tosoh Biosciences) and desalted using reverse phase chromatography performed on AMBERCHROM™ CG300-S resin (Rohm and Haas), and lyophilized.

### Example 2: Improved Synthetic Method for Ultrapure Avacincaptad Pegol

The improved synthetic method that yields the ultrapure Avacincaptad pegol of this presently disclosed subject matter is described below.

The oligonucleotide (ARC672) 5′ NH₂-fCmGfCfCGfCmGmGfUfCfUfCmAmGmGfCGfCfUmGmAmGfUfCfUmGmAmGfUfUfUA fCfCfUmGf CmG-3T-3′ (SEQ ID NO: 1), was synthesized on an OligoPilot 400 (Cytiva life sciences, Marlborough, MA) or other similar synthesizer according to the recommended manufacturer's procedures using commercially available 2′-OMe RNA and 2′-F RNA and TBDMS-protected RNA phosphoramidites (Thermo Scientific, Milwaukee, WI; Hongene Biotech, Shanghai, China; Sigma-Aldrich, Hamburg, Germany) and an inverted deoxythymidine CPG Support (Prime Synthesis, Aston, PA). Terminal amine function was attached with a 5′-amino modifier C6-TFA (Sigma-Aldrich, Hamburg, Germany). After cleavage and deprotection, the oligonucleotides (ARC672) were concentrated and desalted by 5 kDa molecular weight (MW) cut-off Hydrosart membranes (Sartorius Stedim Biotech) or similar membrane from other manufacturers, followed by purification using an ion exchange chromatography with TSK Gel SuperQ-5 PW resin (ToSoh BioSciences). Prior to PEGylation, the purification pool of oligonucleotides (ARC672) is concentrated and desalted via 5 kDa MW cut-off membranes and further concentrated.

The amine-modified aptamer (ARC672) was conjugated to PEG moieties post-synthetically. The concentrated solution of aptamer (ARC672) was diluted in sodium borate buffer (pH 8-10) and DMSO. The aptamer was reacted for less than one hour with less than 1.5 equivalents of the desired PEG reagent (e.g., SUNBRIGHT® GL2-400TS from NOF Corp, Japan) dissolved in DMSO. The resulting product ARC1905 was purified by ion exchange chromatography using TSK Gel SuperQ-5 PW resin (Tosoh Biosciences). The ultrafiltration of the purification pool of ARC1905 was conducted using a 10 kDa molecular weight cutoff membrane to desalt, and then lyophilized.

The improved synthetic method is described in more detail below:

### Stage I: Non-PEGylated ACP Synthesis and Isolation

### 1. Synthesis

Manufacture of avacincaptad pegol began with iterative synthesis of the non-PEGylated avacincaptad pegol (non-PEGylated ACP) on solid support. The oligonucleotide 5′ NH2-fCmGfCfCGfCmGmGfUfCfUfCmAmGmGfCGfCfUmGmAmGfUfCfUmGmAmGfUfUfUA fCfCfUmGf CmG-3T-3′ (SEQ ID NO: 1), was synthesized on an OligoProcess™ oligonucleotide synthesizer (Cytiva Life Sciences, Marlborough, MA) or an oligonucleotide synthesizer from different vendors according to the recommended manufacturer's procedures using commercially available 2′-OMe RNA and 2′-F RNA and TBDMS-protected RNA phosphoramidites and an inverted deoxythymidine CPG Support (LGC BioSearch Technologies, Novato, CA). The oligonucleotide synthesis process comprised of four chemical reactions carried out in the following sequence:
1. Deblocking the protected nucleoside or nascent oligonucleotide (Detritylation)
2. Activation and coupling of the incoming phosphoramidite (Coupling)
3 Oxidation of the resultant phosphite triester P (III) to the P (V) phosphate linkage (Oxidation); and
4. Capping of oligonucleotide chains that failed to successfully couple (Capping)

The four steps above were repeated in sequence until the desired oligonucleotide, terminating in the hexylamino linker, was synthesized. Terminal amine function was attached with a 5′-amino modifier C6-TFA (Sigma-Aldrich, Hamburg, Germany).

The use of 0.2M amidite and 0.5M activator during the synthesis step led to improved purity. Upon completion of the synthesis, the synthesis column was washed with diethylamine to remove the cyanoethyl protecting groups, which also led to improved purity.

### 2. Cleavage and Deprotection

The next steps involved cleavage of non-PEGylated ACP from solid support, removal of base-protecting groups, and deprotection of silyl-protected ribonucleosides. Ammonia and/or alkyl amine base were added to the heated deprotection tank, which was then recirculated through the synthesis column. The cleavage and deprotection reaction mixture was collected. Triethylamine trihydrofluoride (TEA-3HF) was added to the deprotection tank and the mixture was heated to facilitate desilylation and pH adjusted to 6-8.

The use of defined volumes of ammonia and alkyl amine base resulted in less non-PEGylatable and silyl-off species in the deprotection reaction and thus led to improved purity. Performing the desilylation reaction for defined time resulted in increased yield thereby leading to improved purity.

### Stage II: Purification of Non-PEGylated ACP

### 1. Crude Ultrafiltration/Diafiltration

After cleavage and deprotection, crude ultrafiltration/diafiltration was performed to achieve volume reduction and removal of solvents. The crude mixture from cleavage and deprotection step was concentrated and diafiltered using a 5 kDa or 10 kDa nominal molecular weight cut-off (MWCO) Ultrafiltration (UF) Hydrosart membranes (Sartorius Stedim Biotech) or similar membrane from other manufacturers.

### 2. Pre-PEGylation Anion Exchange Chromatography

Anion Exchange (AX) chromatography was next performed to purify the non-PEGylated ACP prior to PEGylation. The crude retentate was loaded onto a chromatography column containing Tosoh Bioscience TSKgel® SuperQ-5 PW chromatography resin or similar resin from other manufacturers. A sodium bromide salt gradient was used to purify the non-PEGylated ACP at a temperature above 45° C.

### 3. Pre-PEGylation Ultrafiltration/Diafiltration (UF/DF2)

A pre-PEGylation ultrafiltration/diafiltration step was then performed to achieve volume reduction and buffer exchange. The purified non-PEGylated ACP solution was concentrated and desalted using a 5 kDa or 10 kDa MWCO ultrafiltration membrane or similar membrane from other manufacturers

### 4. Concentration

The retentate from the previous step was further concentrated prior to PEGylation using vacuum distillation (i.e., Rotary Evaporator, Concentrator) or Thin Film Evaporator to reduce the volume.

### Stage III. PEGylation

A site-specific covalent bond was formed between the primary amine on the 5′ end of the non-PEGylated ACP and the PEGylation reagent (mPEG2-NHS ester) to form the crude drug substance avacincaptad pegol. The non-PEGylated ACP solution was diluted with sodium borate buffer and DMSO and at the pH of 8.8-9.5. The required amount of PEGylation reagent, mPEG2-NHS ester, based on a defined molar ratio to non-PEGylated ACP was dissolved in DMSO and the buffered non-PEGylated ACP solution was then added to initiate PEGylation. After completion, the PEGylation was quenched by adding water.

The mPEG2-NHS ester enhanced the reaction efficiency and reduced process time. The use of 1.1-1.5 equivalents of mPEG2-NHS ester resulted in less residual free PEG to remove, and the PEGylation performed above the room temperature led to decreased thermal exposure, thereby increasing yield and efficiency.

### Stage IV. Purification of Avacincaptad Pegol

### 1. Post-PEGylation Anion Exchange Chromatography

Anion Exchange (AX) chromatography was used to purify the crude drug substance following PEGylation. The crude drug substance from the PEGylation step was loaded onto a chromatography column containing Tosoh Bioscience TSKgel® SuperQ-5 PW chromatography resin or similar resin from other manufacturers. A sodium bromide salt gradient was used to purify the drug substance at a temperature above 45° C.

### 2. Post-PEGylation Ultrafiltration/Diafiltration

A post-PEGylation Ultrafiltration/Diafiltration step was then performed to achieve volume reduction and buffer exchange. The purified avacincaptad pegol was concentrated and desalted using a 10 kDa nominal MWCO membrane or similar membrane from other manufacturers, which led to increased product retention.

### Stage V. Lyophilization

The drug substance solution was filtered and freeze-dried to reduce water content and the product was packaged.

### Example 3: Drug Product

ARC1905 drug product was formulated as a preservative-free, sterile, aqueous solution for intravitreal injection. It was formulated at a concentration of 20 mg/mL (oligonucleotide mass) in phosphate-buffered saline at pH 6.8-7.8 as a sterile aqueous solution. The drug product was stable at 2-8° C. for 43 months.

The osmolality of one batch of the drug product is shown in Table 1 below:

TABLE 1

| Oligonucleotide | | |
|---|---|---|
| Concentration | | Average Osmolality |
| (mg/mL) | Media | (mOsm/kg) (n = 2) |
| 20 | 10 mM PBS pH 7.3 | 429 |

### Example 4: Comparison of Purity Profile of Prior Art and Ultrapure Manufacture Methods

Differences in the purity profile of the ACP product made by the prior art method and the improved method are described in Table 2 and Table 3 below:

TABLE 2

| Pre-PEGylated Aptamer | Prior Art Process | Ranges from Improved |
|---|---|---|
| FLP | 80.9%* | 88.3-91.8% |
| RRT Range 1 | 2.07% | 1.06-1.71% |
| (G cleavage) | | |
| RRT Range 2 | 0.17% | 0-0.08% |
| (A cleavage) | | |
| RRT Range 3 | 1.86% | 0.79-1.05% |
| (n − 4, n − 3, n − 2 deletion) | | |
| RRT Range 4 | 5.45% | 2.12-2.86% |
| (Fluoro Degradants, n − 1 | | |
| deletion) | | |
| RRT Range 5 | 0.66% | 0.38-0.66% |
| (n + 1 Additions) | | |
| RRT Range 6 | 3.94% | 0.55-1.00% |
| (Deprotection Failures) | | |

*FLP may contain ~4% methylation impurities
FLP = full length product
G cleavage = products from cleavage of non-PEGylated ACP at position(s) of G in the sequence
A cleavage = products from cleavage of non-PEGylated ACP at position of A in the sequence

TABLE 3

| ACP | Prior Art Process | Ranges for Improved |
|---|---|---|
| FLP | 90.6%* | 93.0-94.0% |
| RRT 1 (≥0.93-<FLP) | 1.30% | 1.97-2.65% |
| RRT 2 (>FLP-≤1.20) | 8.15% | 3.00-4.74% |

*FLP may contain ~4% methylation impurities

In some embodiments, the content of endotoxin in the ultrapure drug substance is less than 0.2 EU/dose. In one embodiment, the content of endotoxin in the drug product is about 0.14 EU/dose, preferably about 0.05 EU/dose.

### Example 5: Potency of Ultrapure Drug Substance

Potency of the ultrapure intermediate and drug substance was measured by ELISA. The ELISA assay is based on induction of complement cascade by Lipopolysaccharide (LPS) and quantification of C5b9 formation. The higher the potency that the ACP drug substance has, the lower the amount of C5b9 that is detected. The result is expressed as relative potency (%) compared to standard ACP reference material.

Based on the ELISA results, the ultrapure drug substance was at least 5% more potent compared to product made according to the prior art synthesis,

### Example 6: Comparison of Potency of Prior Art and Ultrapure Manufacturing Methods

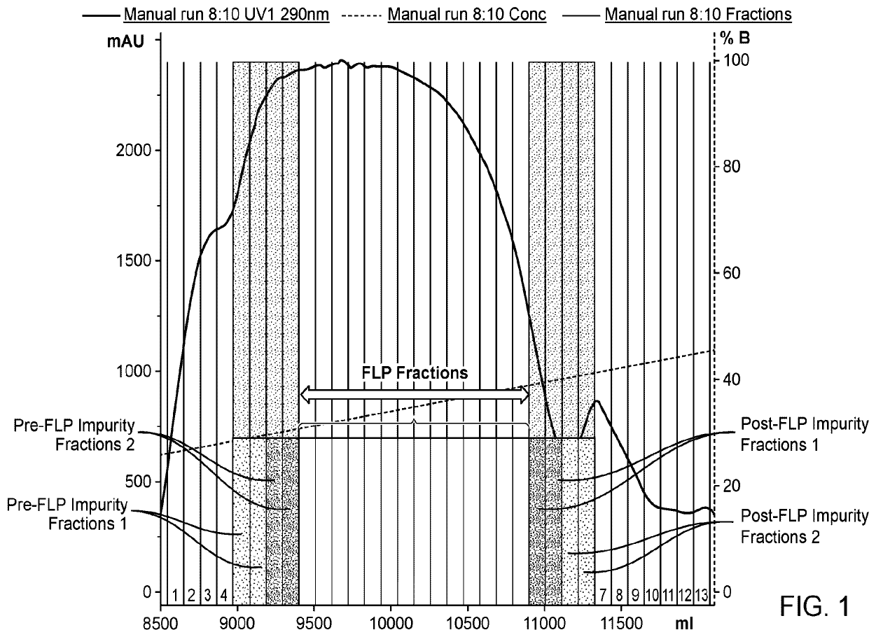

Potency of impurity fractions of the ACP product made by the prior art method and the improved method was measured by ELISA. The impurity fraction collections are shown in FIG. 1. The data comparison is shown in Table 4.

The results show that the pre- and post-FLP impurity fractions from the prior art process have higher potency than those from the improved manufacturing process. This demonstrates that the improved process has a high purification resolution and efficiency.

TABLE 4
Potency data comparison for impurity fractions between the
prior art process and the improved manufacturing process

| Post PEGylation | | |
|---|---|---|
| Purification fractions | Improved Process | Prior Art |
| Pre-FLP impurity Fraction 1 | 12% | 21% |
| Pre-FLP impurity Fraction 2 | 53% | 53% |
| Post-FLP impurity Fraction 1 | 22% | 57% |
| Post-FLP impurity Fraction 2 | 21% | 35% |

1. A composition comprising a non-pegylated aptamer 5′ NH₂-fCmGfCfCGfCmGmGfUfCfUfCmAmGmGfCGfCfUmGmAmGfUfCfUmGmAmGf UfUfUAfCfCfUmGf CmG-3T-3′ (SEQ ID NO: 1), wherein:
(a) more than 85% of the aptamer in the composition is full length aptamer, and wherein the composition comprises:
(b) less than 1.8% of G cleavage products,
(c) 1% or less of deprotection failure products,
(d) less than 0.1% of A cleavage products,
(e) less than 1.1% of n−4, n−3 and n−2 deletion products, and
(f) less than 3% of fluoro degradants and n−1 deletion products.

2. The composition of claim 1, wherein:
(a) 88.3%-91.8% of the aptamer in the composition is full length aptamer; and wherein the composition comprises:
(b) 1.06%-1.71% of G cleavage products,
(c) 0.55%-1.00% of deprotection failure products,
(d) 0%-0.08% of A cleavage products, and
(e) 2.12%-2.86% of fluoro degradants and n−1 deletion products.

3. A composition, comprising a PEGylated aptamer having the structure: 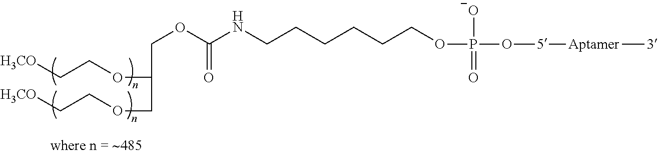
or a salt thereof, wherein the aptamer comprises the sequence of SEQ ID NO: 1, wherein:
(a) more than 92% of the aptamer in the composition is full length aptamer; and
(b) less than 5% of the composition is relative retention time 2 (RRT2) (>full length product (FLP)-≤1.2).

4. The composition of claim 3, wherein:
(a) 93.0%-94.0% of the aptamer in the composition is full length aptamer,
(b) 3.00%-4.74% of the composition is RRT 2 (>FLP-≤1.20), and
(c) 1.97%-2.65% of the composition is RRT 1 (≥0.93-<FLP).

5. The composition of claim 3, wherein the PEGylated aptamer comprises a 2-arm branched PEG ranging from approximately 39 kDa to approximately 47 kDa.

6. The composition of claim 3, wherein the PEGylated aptamer comprises a 2-arm branched PEG of approximately 40 kDa.

7. The composition of claim 3, wherein the PEGylated aptamer comprises a 2-arm branched PEG of approximately 43 kDa.

8. The composition of claim 3, wherein the salt is a sodium salt.

9. The composition of claim 8, wherein the composition is at least 5% more potent at inducing a complement cascade as measured by ELISA, as compared to a control composition, wherein:
(a) 90.6% of the aptamer in the control composition is full length product,
(b) 1.3% of the control composition is RRT 1 (≥0.93-<FLP), and
(c) 8.15% of the control composition is RRT 2 (>FLP-≤1.20).

10. The composition of claim 3, comprising one or more pharmaceutically acceptable excipients.

11. The composition of claim 3, wherein the composition is a sterile aqueous solution comprising 20 mg/mL of the aptamer and phosphate-buffered saline and having a pH in the range of 6.8-7.8.

12. The composition of claim 11, wherein the composition has a pH of 7.3.

13. The composition of claim 3, wherein the composition has an osmolality between 350 mOsM/kg to 500 mOsM/kg.

14. A method for treating geographic atrophy secondary to age-related macular degeneration, the method comprising intravitreally administering to a subject in need thereof, the composition of claim 3 at a dose between about 1 mg/eye to about 5 mg/eye.

15. The method of claim 14, comprising administering the composition at a dose of about 2 mg/eye.

16. The method of claim 14, comprising injecting about 100 μL of the composition per eye.

17. The method of claim 14, wherein the dose is administered once monthly.

18. The method of claim 14, wherein the dose is administered once every 28±7 days.
